(19) United States
(12) Patent Application Publication
(10) Pub. No.: US 2021/0332406 A1
(43) Pub. Date: Oct. 28, 2021

(54) SEROTYPE DETERMINATION METHOD

(71) Applicant: SHIMADZU CORPORATION, Kyoto-shi (JP)

(72) Inventor: Koichi KOJIMA, Kyoto-shi (JP)

(73) Assignee: SHIMADZU CORPORATION, Kyoto-shi (JP)

(21) Appl. No.: 17/234,069

(22) Filed: Apr. 19, 2021

(30) Foreign Application Priority Data
Apr. 27, 2020 (JP) 2020-077983

(51) Int. Cl. C12Q 1/04; G01N 27/64

(52) U.S. Cl. CPC C12Q 1/04; C12R 2001/19; G01N 2333/245; G01N 27/64

(57) ABSTRACT

The present invention provides a novel serotype determination method for E. coli and the like which is not subject to the limitations of indirect methods such as antigen-antibody reaction and gene analysis.

For example, provided is a method of determining a serotype of E. coli, comprising the following steps 1 to 3:
1. a step of selecting a first peak group consisting of a plurality of peaks which are successively and repeatedly detected at equal intervals in a spectrum obtained by performing a mass spectrometry on a sample containing an antigenic site obtained from a microbial specimen of a determination subject, and then obtaining a difference in m/z value between adjacent peaks of the first peak group as a peak interval actual measurement m/z value of the first peak group, p0 2. a step of collating the peak interval actual measurement m/z value of the first peak group with a separately obtained m/z value for the basic structure corresponding to a specific serotype, and
3. a step of determining the specific serotype corresponding to the separately obtained m/z value for the basic structure consistent with the peak interval actual measurement m/z value of the first peak group, as a serotype of the microorganism, from the result of the above-mentioned collation step.

### TECHNICAL FIELD

The present invention relates to a serotype determination method. Specifically, the present invention relates to a method of determining a serotype or an antigen type of a given microorganism by mass spectrometry.

### BACKGROUND OF THE INVENTION

For example, food poisoning caused by pathogenic Escherichia coli can result in thousands of infected people per year and death in infants and the elderly, in Japan. Therefore, food poisoning is a disease with extremely serious symptoms, and determining the serotype of the causative microorganisms is important for diagnosis, treatment, and identification of the causative foods.

Serotypes of microorganisms are determined by the type of antigens (molecular structure) present on their cell surface. In E. coli, H antigens based on the structure of flagella, O antigens based on the structure of lipopolysaccharides, and the like are known. The lipopolysaccharides are composed of lipids and sugar chains, and the sugar chains are composed of a core polysaccharide moiety and a moiety called O side chain polysaccharides. These O side chain polysaccharides are responsible for the antigenicity of the O type, and have a structure in which a basic structure composed of a combination of 3 to 5 monosaccharides is repeated about 4 to 40 times.

As a method of determining a serotype of microorganisms, a slide aggregation method, a latex aggregation method (e.g., Patent Document 1), other aggregation methods (e.g., Patent Document 2), an immunochromatographic method, and the like have been widely used, but all these methods are based on an antigen-antibody reaction. Therefore, they are sometimes called an antigen-antibody reaction, an immune reaction, or a serum reaction.

Differences in serotypes are, namely, differences in the structure of the antigenic site of the antigenic molecule. The type of O antigen is responsible for the sugar chain structure of lipopolysaccharides present on the surface of the bacterial cell, but unlike proteins, it is not directly encoded by genes. However, since it depends on the sugar chain synthase encoded by a gene, the method of determining the type of the antigen by analyzing the gene has also been put into practical use.

It has also been reported that the serotype of E. coli was determined by mass spectrometry (e.g., Non-Patent Document 1). In this document, a matrix-assisted laser desorption ionization time-of-flight mass spectrometer (MALDI-TOF MS) has been used to rapidly determine the type of H-antigen in E. coli. The methods in this document are based on peptide mass fingerprinting, in which the proteins of E. coli flagella are broken down into peptide fragments (tryptic digestion) and their masses are measured by MALDI-TOF MS.

Methods of identifying microorganisms by mass spectrometry are also known (Patent Document 3). The method of Patent Document 3 is a method of mass-analyzing a sample containing a microorganism, reading an m/z value of a peak derived from a marker protein on the obtained mass spectrum, and determining whether or not the sample contains O157, O26, or O111 based on the m/z value, characterized in that at least either one of a ribosomal protein S15 and a ribosomal protein L25, as well as an acid stress chaperone HdeB and a DNA binding protein H-NS are used as the marker proteins.

### PRIOR ART DOCUMENTS

### Patent Document

Patent Document 1: JP-A-2002-119297

Patent Document 2: JP-B-5142725

Patent Document 3: JP-A-2015-184020

### NON-PATENT DOCUMENT

Non-Patent Document 1: Huixia Chui, et al., Journal of Clinical Microbiology, August 2015, Volume 53, Number 8, pp.2480-2485

### SUMMARY OF THE INVENTION

### Problem to be Solved by the Invention

In the serotype test of a microorganism, as described above, a method based on an antigen-antibody reaction such as a latex aggregation method is widely utilized. This is reasonable because serotype is defined as to react with a particular antibody. However, since one antibody corresponds to one serotype, when a number of serotypes are considered possible, the same number of antibodies must be prepared. For example, when any one of seven serotypes: O157, O26, O111, O103, O121, O145, and O165 is possible for the O group, seven types of antibodies should be used. Moreover, since the activity of an antibody decreases over time, long-term storage is not possible, and it is a large cost to stockpile a variety of antibodies in a usable state.

In the meantime, there is no problem of preserving an antibody in the method based on the gene analysis. However, it is also necessary to prepare a number of reaction assays that correspond to possible types. Moreover, since an antigen is produced as a result of an action of a plurality of genes, there is a problem that the presence of a relevant gene does not necessarily lead to the specific type. In addition, it is not possible to test without the information of the gene which determines the serotype. Therefore, if a new serotype appears, the reaction assay cannot be designed until the relevant gene is analyzed.

The E. coli serotype has also been determined by mass spectrometry, but it is directed against the H antigen, and the analytical method is also based on peptide mass fingerprinting for protein analysis.

The present invention is primarily directed to providing a novel serotype determination method for a certain microorganism which is not subject to the above-mentioned restrictions found in the case of an indirect method such as an antigen-antibody reaction or a genetic analysis. Alternatively, the present invention is primarily directed to providing a novel serotype determination method capable of obtaining test results with higher accuracy by a simple method while avoiding restrictions such as the necessity of multiple types of antibodies and multiple types of assays for gene analysis with respect to certain microorganisms.

### Means for Solving the Problems

As a result of intensive studies, the present inventor has found that the above problem can be solved by directly analyzing a molecular structure contributing to a difference in antigenicity by mass spectrometry, and the present invention was completed.

The present invention can include, for example, the following embodiments.
[1] A method of determining a serotype of a microorganism wherein a sugar chain moiety of a lipopolysaccharide forming an antigen is repeated multiple times with a basic structure composed of a combination of a plurality of monosaccharides as one unit, comprising the following steps 1 to 3:
1. a step of selecting a first peak group consisting of a plurality of peaks which are successively and repeatedly detected at equal intervals in a spectrum obtained by performing a mass spectrometry on a sample containing an antigenic site obtained from a microbial specimen of a determination subject, and then obtaining a difference in m/z value between adjacent peaks of the first peak group as a peak interval actual measurement m/z value of the first peak group,
2. a step of collating the peak interval actual measurement m/z value of the first peak group with a separately obtained m/z value for the basic structure corresponding to a specific serotype, and
3. a step of determining the specific serotype corresponding to the separately obtained m/z value for the basic structure consistent with the peak interval actual measurement m/z value of the first peak group, as the serotype of the microorganism, from the result of the collation step.
[2] An apparatus for determining a serotype of a microorganism wherein a sugar chain moiety of a lipopolysaccharide forming an antigen is repeated multiple times with a basic structure composed of a combination of a plurality of monosaccharides as one unit, comprising the following units 1 to 4:
1. a data acquisition unit, which acquires a mass spectrum data obtained by performing a mass spectrometry on a sample containing an antigenic site prepared from a microbial specimen of a determination subject,
2. a first peak interval acquisition unit, which selects a first peak group consisting of a plurality of peaks which are successively and repeatedly detected at equal intervals in the mass spectrum obtained in the above-mentioned data acquisition unit, and then obtains a difference in m/z value between adjacent peaks of the first peak group as a peak interval actual measurement m/z value of the first peak group,
3. a data collation unit, which collates the actual measurement m/z value with a separately obtained m/z value for the basic structure corresponding to a specific serotype, and
4. a serotype determination section, which determines a specific serotype corresponding to the separately obtained m/z value consistent with the actual measurement m/z value, as the serotype of the microorganism.
[3] A program for determining a serotype of a microorganism wherein a sugar chain moiety of a lipopolysaccharide forming an antigen is repeated multiple times with a basic structure composed of a combination of a plurality of monosaccharides as one unit, executing the steps comprising the following 1 to 4:
1. a step of performing a mass spectrometry on a sample containing an antigenic site prepared from a microbial specimen of a determination subject, and then acquiring an interval between each adjacent peak of a plurality of peaks detected successively and repeatedly at equal intervals in a spectrum obtained therein as an actual measurement m/z value,
2. a step of collating the actual measurement m/z value with a separately obtained m/z value for the basic structure corresponding to a specific serotype,
3. a step of determining the specific serotype corresponding to the separately obtained m/z value consistent with the actual measurement m/z value, as the serotype of the microorganism, and
4. a step of outputting the determined serotype of the microorganism.

### Effect of the Invention

According to the present invention, it is possible to determine the serotype without requiring an antibody corresponding to the serotype to be tested, a primer for gene analysis, or the like. Samples can be prepared in a similar manner regardless of serotypes, and serotypes can be determined under the same conditions regardless of serotypes.

In the conventional method, a plurality of reactions is performed on 1 sample, but according to the present invention, since a difference in a serotype appears as a difference in spectrum of mass spectrometry, one measurement may be performed on one sample. Moreover, although the conventional method cannot analyze a new serotype, the present invention can analyze the new serotype, and in the present method, even if a mass spectrum that has not been reported is observed, it can be found that the mass spectrum is derived from a new serotype. In addition, in the present method, it can be found that even those with the same serotype in the conventional method have different sugar chain structures.

### BRIEF DESCRIPTION OF THE DRAWINGS

FIG. 1 represents a mass spectrum of lipopolysaccharide from E. coli O157.

FIG. 2 represents the mass spectrum of FIG. 1 to which m/z values of peak intervals are added.

FIG. 3 represents a mass spectrum of a sample.

FIG. 4 represents a mass spectrum of reference sample A.

FIG. 5 represents a mass spectrum of reference sample B.

FIG. 6 represents a mass spectrum of reference sample C.

FIG. 7 represents a result of subjecting reference sample A to immunochromatography.

FIG. 8 represents a result of subjecting reference sample B to immunochromatography.

FIG. 9 represents a result of subjecting reference sample C to immunochromatography.

FIG. 10 represents a flowchart illustrating an embodiment of the serotype determination system or the like according to the present invention.

FIG. 11 represents a flowchart illustrating an embodiment of the serotype determination program according to the present invention.

FIG. 12 represents a result of subjecting sample O111 to immunochromatography.

FIG. 13 represents a result of subjecting sample O111* to immunochromatography.

FIG. 14 represents a mass spectrum of sample O111.

FIG. 15 represents a mass spectrum of sample O111*.

### EMBODIMENT FOR CARRYING OUT THE PRESENT INVENTION

Hereinafter, the present invention will be described in detail.

### A. Serotype Determination Method According to the Present Invention

The serotype determination method according to the present invention (hereinafter, referred to as “the present invention determination method”) is a method of determining a serotype of a microorganism wherein a sugar chain moiety of a lipopolysaccharide forming an antigen is repeated multiple times with a basic structure composed of a combination of a plurality of monosaccharides as one unit, comprising the following steps 1 to 3:
1. a step of selecting a first peak group consisting of a plurality of peaks which are successively and repeatedly detected at equal intervals in a spectrum obtained by performing a mass spectrometry on a sample containing an antigenic site obtained from a microbial specimen of a determination subject, and then obtaining a difference in m/z value between adjacent peaks of the first peak group as a peak interval actual measurement m/z value of the first peak group,
2. a step of collating the peak interval actual measurement m/z value of the first peak group with a separately obtained m/z value for the basic structure corresponding to a specific serotype, and
3. a step of determining the specific serotype corresponding to the separately obtained m/z value for the basic structure consistent with the peak interval actual measurement m/z value of the first peak group, as the serotype of the microorganism, from the result of the collation step.

A microorganism targeted by the present invention has a molecule having a structure which is repeated multiple times with a basic structure composed of a combination of multiple monosaccharides as one unit, and the basic structure may compose a sugar chain moiety of a lipopolysaccharide forming an antigen of a microorganism of a determination subject. The microorganism is not particularly limited as long as having such a structural molecule. The microorganism with such structural molecules may include, for example, Gram-negative bacteria, Gram-negative facultative anaerobic bacilli, Gram-negative aerobic bacilli, Gram-negative anaerobic bacilli, and Gram-negative cocci. Specifically, the following bacteria can be exemplified.
Gram-negative facultative anaerobic bacilli Escherichia coli (Eshericha coli), Shigella spp. (Shigella), Salmonella spp. (Salmonella), Klebsiella spp. (Klebsiella), Proteus spp. (Proteus), Yersinia spp. (Yersinia), Vibrio cholerae (V. cholerae), Vibrio parahaemophilus spp. (Vparahaemolyticus), and Haemophilus spp. (Haemophilus).
Gram-negative aerobic bacilli Pseudomonas spp. (Pseudomonas), Legionella spp. (Legionella), Bordetella spp. (Bordetella), Brucella spp. (Brucella), Tularemia spp. (Francisella tularensis)
Gram-negative anaerobic bacilli Bacteroides (Bacteroides)
Gram-negative cocci Neisseria spp. (Neisseria)

Especially, the present invention is useful for E. coli of group O. As for O-Antigen Escherichia coli about 180 kinds are currently known, and pathogenic O antigen Escherichia coli can include, for example, EPEC (enteropathogenic Escherichia coli), EAEC (enteroaggregative Escherichia coli), EHEC (enterohemorrhagic Escherichia coli), ETEC (enterotoxigenic Escherichia coli), EIEC (enteroinvasive Escherichia coli), DAEC (diffusely adherent Escherichia coli), and UPEC (uropathogenic Escherichia coli) MNEC (meningitis/sepsis-associated Escherichia coli). Specifically, EPEC can include O18ac, O20, O25, O26, O28, O44, O55, O86, O91, O111, O114, O119, O125ac, O126, O127, O128, O142, O146, O151, O158, O159, and O166, for example. ETEC can include O6, O8, O11, O15, O20, O25, O27, O63, O73, O78, O85, O114, O115, O128, O139, O148, O149, O159, O166, O167, O168, O169, and O173, for example. EIEC can include O6, O28ac, O29, and O112ac, O115, O124, O136, O143, O144, O152, O159, O164, P167, for example. EHEC can include O1, O4, O5, O8, O16, O18, O25, O26, O44, O46, O48, O55, O86, O91, O98, O103, O111ab, O113, O114, O115, O117, O118, O119, O124, O125, O126, O127, O128, O145, O153, O157, O166, O167, O169, O172, O176, O177, O178, O179, O180, and O181, for example. EAEC can include O3, O7, O15, O44, O55, O77, O78, O86, O111, O125, O126, O127, O128, and O157. UPEC includes O4, O6, O14, O22, O75, and O83, for example.

The monosaccharide of the sugar chain moieties of microorganisms or O-antigen E. coli may include colitose, fucose, N-acetylfucosamine, glucose, glucuronic acid, N-acetylglucosamine, galacturonic acid, N-acetylgalactosamine, N-acetylgalacturonic acid, mannose, N-acetylneuraminic acid, N-acetylquinobosamine, rhamnose, ribose, 6-deoxytalose, N-acetylperosamine, glucosamine, galactosamine, fucosamine, talose, neuraminic acid, galactosamine, xylulose, linose, fucose, fluranose, furose, fucose, flucose, fucose, flucopic acid, fluorose, fluorose, and fluopyrose, for example. These monosaccharides may be D bodies, L bodies, or anomers.
A-1 Re: Step 1

Step 1 is a step of selecting a first peak group consisting of a plurality of peaks which are successively and repeatedly detected at equal intervals in a spectrum obtained by performing a mass spectrometry on a sample containing an antigenic site obtained from a microbial specimen of a determination subject, and then obtaining a difference in m/z value between adjacent peaks of the first peak group as a peak interval actual measurement m/z value of the first peak group.

In carrying out the present invention, first, a sample containing a molecular structural moiety or a structural moiety thereof contributing to antigenicity from a microorganism of a determination subject is prepared. Although depending on the analytical method of the next step, it is preferable that the sample is prepared so that a molecule having an antigenic site becomes a main component, for example, when a MALDI-TOF MS is used. Here, the term “main component” means that other components may be included within a range not impairing the effect of the present invention, and does not limit the inclusion of other components, but usually means that the content ratio of the molecule containing the antigen site to the entire component accounts for 50% by weight or more. Preferably, the content is 70% by weight or more, more preferably 80% by weight or more to 90% by weight or more. The content may be 100% by weight.

Specifically, for example, when the type of O antigen of E. coli tested, lipopolysaccharide (glycolipid) contributing to antigenicity is prepared as a main component by a conventional method. The conventional methods include, but are not limited to, a phenol extraction method and an acetone precipitation method, for example. Alternatively, in lipopolysaccharides, a molecular structural moiety contributing to antigenicity is a portion of an O side chain polysaccharide, and therefore, only a sugar chain moiety may be prepared by removing the lipid moiety from the lipopolysaccharide in a cleavage method such as acid hydrolysis.

Next, the sample obtained as described above is measured by mass spectrometer. This mass spectrometer is not particularly limited as long as it includes at least an ionization unit and a mass separation unit, and is capable of measuring an m/z value of charged particles.

Although the ionization method in the ionization unit is not particularly limited, since the main sample is a sugar chain, from the viewpoint of easily ionizing in a state of keeping the molecular structure, it is desirable to use a soft ionization method. The ionization method can include FAB (Fast Atom Bombardment: fast atomic bombardment method), MALDI (Matrix-Assisted Laser Desorption/Ionization (matrix-assisted laser desorption ionization method), ESI (Electrospray Ionization: electrospray ionization method), PESI (Probe Electrospray Ionization: probe electrospray ionization method), APCI (Atmospheric Pressure Chemical Ionization: atmospheric pressure chemical ionization method), DIOS (Desorption/Ionization on Silicon: desorption ionization from porous silicon), and SALDI (Surface-Assisted Laser Desorption/Ionization: surface-assisted laser desorption ionization method), for example. From the viewpoint of easily generating fragment ions for contributing to the analysis of the molecular structure, it is also possible to collide an inert gas molecule such as a rare gas (He, Ne, Ar, or the like) with respect to charged particles generated by soft ionization, by CID (Collision-Induced Dissociation: collision-induced dissociation method).

Moreover, the mass separation method in the mass separation unit is also not limited. A magnetic field type mass spectrometer, a quadrupole mass spectrometer (Q MS: Quadrupole Mass Spectrometer), an ion trap mass spectrometer (IT MS: on Trap Mass Spectrometer), a time-of-flight mass spectrometer (TOF MS: Time-of-Flight Mass Spectrometer), a Fourier transform ion cyclotron resonance mass spectrometer (FT-ICR MS: Fourie Transform-Ion Cyclotron Resonance Mass Spectrometer), or an orbitrap (orbitrap) can be used, for example.

Furthermore, as the mass spectrometer, any combination of the ionization method and the mass separation method described above may be used. For example, but not limited to, MALDI-TOF MS (matrix-assisted laser desorption ionization-time-of-flight mass spectrometer), ESI-TOF MS (electrospray ionization-time-of-flight mass spectrometer), APCI-TOF MS (atmospheric pressure chemical ionization-time-of-flight mass spectrometer), ESI-IT MS (electrospray ionization-ion trap mass spectrometer), APCI-IT MS (atmospheric pressure chemical ionization-ion trap mass spectrometer), FAB-IT MS (fast atom bombardment-ion trap mass spectrometer), ESI-Q MS (electrospray ionization-quadrupole mass spectrometer), PESI-Q MS (probe electrospray ionization-quadrupole mass spectrometer), or APCI-Q MS (atmospheric pressure chemical ionization-quadrupole mass spectrometer) can be used.

The mass spectrometer in the present invention may be a single mass spectrometer or a device of tandem mass spectrometry (Mass Spectrometry/Mass Spectrometry: MS/MS). Multistage tandem-mass spectrometry (MSⁿ) equipment can also be used.

Moreover, the mass spectrometer in the present invention may include other device units having functions other than those described above. The device units can include a chromatograph and a microscope, for example. Accordingly, the mass spectrometer in the present invention can include a liquid chromatograph mass spectrometer (LC/MS), a supercritical fluid chromatograph mass spectrometer (SFC/MS), an imaging mass microscope, and the like.

In general, in the interior of a mass spectrometer, a sample can be ionized while keeping a molecular structure as it is, but it is usual that fragment ions derived from a decomposition product of a sample are also generated together. Degradation (fragmentation) of the sample also occurs until the ionized sample is detected in the device, or may also occur prior to ionization or in parallel with ionization.

According to the above, a mass spectrum consisting of a plurality of peaks corresponding to m/z values of ions derived from molecules contained in a sample is obtained. These peaks include those derived from molecular structural moieties contributing to antigenicity. For example, since in the O antigen of Escherichia coli a unit composed of 3 to 5 monosaccharides is repeated, a plurality of peaks of intervals derived from the unit are observed. Moreover, a peak derived from the molecular weight of the monosaccharide composing the unit is also observed.

In the present invention, a peak interval derived from one unit of a basic structure composed of a combination of multiple monosaccharides, that is, a size of an adjacent peak interval of the first peak group is obtained as an actual measurement value of m/z, and this is utilized.

Moreover, the size (m/z value) of the peak interval derived from the molecular weight of the monosaccharide composing the basic structure can be similarly utilized. Here, a peak group composed of peaks derived from multiple monosaccharide molecular weights detected between each peak composing the first peak group is referred to as a “second peak group”.
A-2 Re: Steps 2 and 3

Step 2 is a step of collating the peak interval actual measurement m/z value of the first peak group with a separately obtained m/z value for the basic structure corresponding to a specific serotype. Step 3 is a step of determining the specific serotype corresponding to the separately obtained m/z value for the basic structure consistent with the peak interval actual measurement m/z value of the first peak group, as the serotype of the microorganism, from the result of the above-mentioned collation step.

In the present invention, for some specific serotypes, the size of the peak interval derived from one unit of the basic structure composed of a combination of multiple monosaccharides is obtained in advance as a predetermined value of m/z. These and the peak interval actual measurement m/z value of the first peak group obtained in the above-mentioned measurement can be collated to determine the corresponding one as the serotype of the microorganism.

In addition, the size (m/z) of the peak interval derived from the molecular weight of the monosaccharide composing the basic structure can also be collated to increase the accuracy of serotype determination. Therefore, the present invention may further include, for example, a step of collating a peak interval actual measurement m/z value between a peak of a second peak group, and a peak of the first peak group adjacent thereto or a peak of the second peak group, with a separately obtained peak interval predetermined m/z value for a detected peak group derived from molecular weights of monosaccharides composing the basic structure corresponding to a specific serotype, or with a peak interval predetermined m/z value separately obtained by calculation for the peak group derived from molecular weights of monosaccharides composing the basic structure, and adding the collating result as a condition for determining a serotype.

The separately obtained peak interval predetermined m/z value for the basic structure corresponding to a specific serotype can be obtained, for example, by calculating based on the structure when the basic structure contributing to antigenicity is known. For example, in the O antigen of E. coli O15 the repeating unit is composed of three monosaccharides, galactose, acetylcosamine, and acetylglucosamine, so that the m/z value is the sum of the respective molecular weights minus the molecular weight of three water molecules which are lost by the glycosidic bond. Specifically, it becomes m/z 552.5 (180.155+205.208+221.207−3×18.015=552.53).

Moreover, the size (m/z value) of the peak interval derived from the molecular weight of the monosaccharide composing the basic structure in which the sugar chain moiety of the lipopolysaccharide forming the antigen is composed of a combination of multiple monosaccharides can be also calculated. For O15, galactose-derived m/z is 162. 1 (180.155−18.015=162.140); acetylfucosamine-derived m/z is 187. 2 (205.208−18.015=187.193); and acetylglucosamine-derived m/z is 203. 2 (221.207−18.015=203.192).

Once calculated, the calculation result can be used as the predetermined value from the second time. It is useful to compile the calculation results into a database. In addition, a value calculated by others can be used as the predetermined value. It is also possible to use databases constructed by others.

Moreover, the predetermined value can be obtained by using a microorganism having a known serotype. For example, a mass spectrum of a microorganism whose serotype is known through a method using an antibody or the like is obtained in a method similar to that of the present invention, and a peak interval m/z value of successive equally spaced repeating peaks observed in the mass spectrum can be obtained. Peak intervals derived from monosaccharide molecular weights can also be obtained as m/z values. These values can be used as predetermined values for the peak interval of the serotype.

The present invention also includes the present invention determination method, further comprising the steps of performing the above-mentioned steps 1 to 3 on a microbial specimen of known serotype, obtaining a peak interval m/z value for the successive equally spaced repeating peaks seen in the resulting mass spectrum, and using such m/z value as the predetermined value.

Once measured, the value can be used repeatedly. It is useful to compile the measured values into a database. It is also possible to use values measured by others, or databases constructed through measurement by others.

Furthermore, without numerical values, it is possible to determine a serotype even by directly collating the mass spectra with each other, and carry out the present invention determination method.

### B. Serotype Determination Apparatus According to the Present Invention

The serotype determination apparatus according to the present invention (hereinafter referred to as “the present invention determination apparatus”) is an apparatus for determining a serotype of a microorganism wherein the sugar chain moiety of a lipopolysaccharide forming an antigen is repeated multiple times with a basic structure composed of a combination of multiple monosaccharides as one unit, characterized by comprising the following units 1 to 4:
1. a data acquisition unit, which acquires a mass spectrum data obtained by performing a mass spectrometry on a sample containing an antigenic site prepared from a microbial specimen of a determination subject,
2. a first peak interval acquisition unit, which selects a first peak group consisting of a plurality of peaks which are successively and repeatedly detected at equal intervals in the mass spectrum obtained in the above-mentioned data acquisition unit, and then obtains a difference in m/z value between adjacent peaks of the first peak group as a peak interval actual measurement m/z value of the first peak group,
3. a data collation unit, which collates the actual measurement m/z value with a separately obtained m/z value for the basic structure corresponding to a specific serotype, and
4. a serotype determination unit, which determines a specific serotype corresponding to the separately obtained m/z value consistent with the actual measurement m/z value, as the serotype of the microorganism.

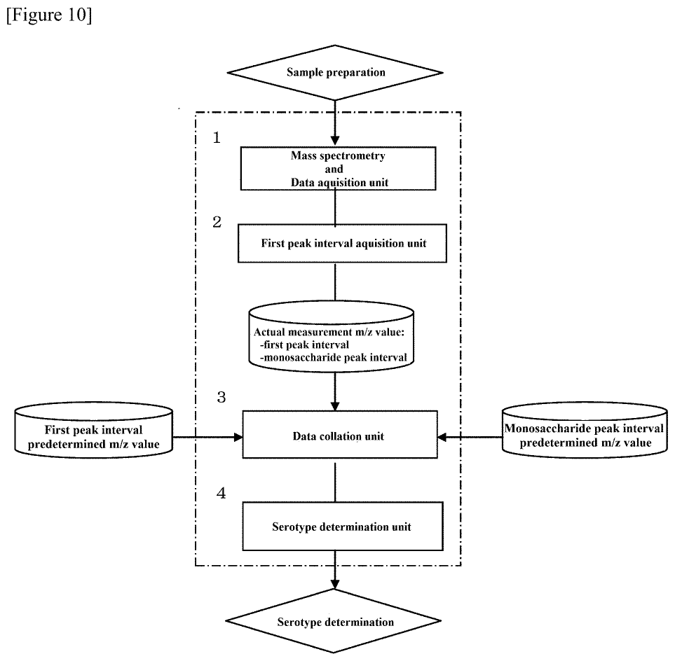

An embodiment of the present invention determination apparatus can be represented by a flowchart as shown in FIG. 10, for example.

A sample containing an antigenic site prepared from a microbial specimen of a determination subject can be obtained, for example, as described in the above-mentioned Step 1 according to the present invention determination method.

The apparatus capable of analyzing the prepared sample by mass spectrometry is not particularly limited as long as it includes at least an ionization means and a mass separation means, and includes a means capable of measuring m/z of charged particles.

Although the ionization method in the ionization means is not particularly limited, since the main sample is a sugar chain, from the viewpoint of easily ionizing in a state of keeping the molecular structure, it is desirable to use a soft ionization method. The ionization methods include FAB, MALDI, ESI, PESI, APCI, DIOS, SALDI, for example. From the viewpoint of facilitating generation of fragment ions to contribute to analysis of the molecular structure, CID may be used.

Moreover, the mass separation method in the mass separation means is also not limited. For example, a magnetic field type mass spectrometer, Q MS, IT MS, TOF MS, FT-ICR MS, or an orbitrap can be used.

Furthermore, as the mass spectrometry means, any combination of the ionization method and the mass separation method described above may be used. For example, but not limited to, MALDI-TOF MS, ESI-TOF MS, APCI-TOF MS, ESI-IT MS, APCI-IT MS, FAB-IT MS, ESI-Q MS, PESI-Q MS, and APCI-Q MS may be used.

The mass spectrometry means may be a single-MS, a MS/MS, or MSⁿ. Moreover, it may further include other means having a function such as a chromatograph or a microscope. Therefore, the mass spectrometry means may be LC/MS, SFC/MS, imaging mass microscopy, or the like.

The apparatus units 1 to 4 include, for example, an information processing apparatus such as a computer, which is housed inside the apparatus having the above-mentioned means, or which is connected to the outside by wire or wirelessly. Among these units, first, a mass spectrum consisting of a plurality of peaks corresponding to the mass and charge of molecules contained in a sample is obtained by the data acquisition unit. Then, a peak group consisting of a plurality of peaks successively and repeatedly detected at equal intervals observed in the obtained spectrum is selected as a first peak group, and a difference in the value of m/z between adjacent peaks of the first peak group, that is, a repeating unit is acquired as a peak interval actual measurement m/z value of the first peak group by the first peak interval acquisition unit. For example, since a unit composed of 3 to 5 monosaccharides is repeated in the O antigen of E. coli, the structural unit becomes the first peak group, and the m/z difference between the peaks derived therefrom is obtained as an actual measurement value. Moreover, the data acquisition unit can also acquire the m/z value of the peak derived from the molecular weight of the monosaccharide composing the unit and the peak interval m/z value.

Next, in the present invention determination apparatus, the actual measurement value obtained above and the predetermined value obtained separately are collated by the data collation unit which collates the peak interval actual measurement m/z value of the first peak group with the predetermined value of the m/z obtained separately for the basic structure corresponding to a specific serotype. The predetermined value can be obtained, for example, in the same manner as described above. Then, the serotype of a microorganism is determined through a serotype determination unit that determines a serotype according to the predetermined value consistent with the actual measurement value among the predetermined values, as the serotype of the microorganism. Furthermore, the size of the peak interval derived from the monosaccharide can also be collated by the data collation unit to improve the accuracy of the serotype determination.

The present invention determination apparatus may include the following apparatus units 5 and/or 6:
5. a data collation unit, which collates a peak interval actual measurement m/z value between a peak of the peak group consisting of a plurality of monosaccharide molecular weight-derived peaks detected between each peak composing the first peak group and a peak of the first peak group adjacent thereto or a monosaccharide molecular weight-derived peak, with a peak interval predetermined m/z value separately obtained for a detected peak group derived from molecular weights of monosaccharides composing the basic structure corresponding to a specific serotype or with a predetermined m/z value separately obtained by calculation for molecular weights of monosaccharides composing the basic structure, and/or
6. a serotype determination unit, which can add the collation result as a condition for the serotype determination.

The data collation unit 5 and the serotype determination unit 6 may serve as the data collation unit 3 and the serotype determination unit 4, respectively, or may be separate apparatus units.

Each of the apparatus units 1 to 6 may be independent of each other or may be any combination of two or more units. For example, the above-mentioned apparatus unit 1 and the above-mentioned apparatus unit 2 may be merged into one apparatus unit. Alternatively, all of the above-mentioned apparatus units 1 to 6 may be merged into one apparatus unit.

The microorganism and the serotype targeted by the present invention determination apparatus are the same as those targeted by the present invention determination method, and are as described above.

### C. Serotype Determination Programs According to the Present Invention

The serotype determination program according to the present invention (hereinafter referred to as “the present invention determination program”) is a program for determining a serotype of a microorganism wherein the sugar chain moiety of a lipopolysaccharide forming an antigen is repeated multiple times with a basic structure composed of a combination of multiple monosaccharides as one unit, executing the steps comprising the following 1 to 4:
1. a step of performing a mass spectrometry on a sample containing an antigenic site prepared from a microbial specimen of a determination subject, and then acquiring an interval between each adjacent peak of a plurality of peaks detected successively and repeatedly at equal intervals in a spectrum obtained therein as an actual measurement m/z value,
2. a step of collating the actual measurement m/z value with a separately obtained m/z value for the basic structure corresponding to a specific serotype,
3. a step of determining the specific serotype corresponding to the separately obtained m/z value consistent with the actual measurement m/z value, as the serotype of the microorganism, and
4. a step of outputting the determined serotype of the microorganism.

The present invention determination program is a program for executing the above-mentioned present invention determination apparatus.

The present invention determination program specifically realizes the serotype determination of the above-mentioned microorganism with hardware resources such as mass spectrometry means, data processing means, storage medium, output means and the like, and may be incorporated in any of the above-mentioned or other hardware resources or installed therein. Therefore, the present invention determination program includes any one of firmware, middleware, application software, and the like.

In the present invention determination program, a program corresponding to an interface between the present invention determination apparatus and the operator or the like can be usually executed by a computer included in the present invention determination apparatus. It may be executed by a computer not included in the present invention determination apparatus. The computer is not particularly limited as long as it includes a processor for executing operations, data processing, and the like, and includes a minimum input unit and a storage medium necessary for the operation of the present invention determination apparatus, and may be a desktop type or a notebook type, and may be a tablet terminal, a smartphone, a wearable, or the like.

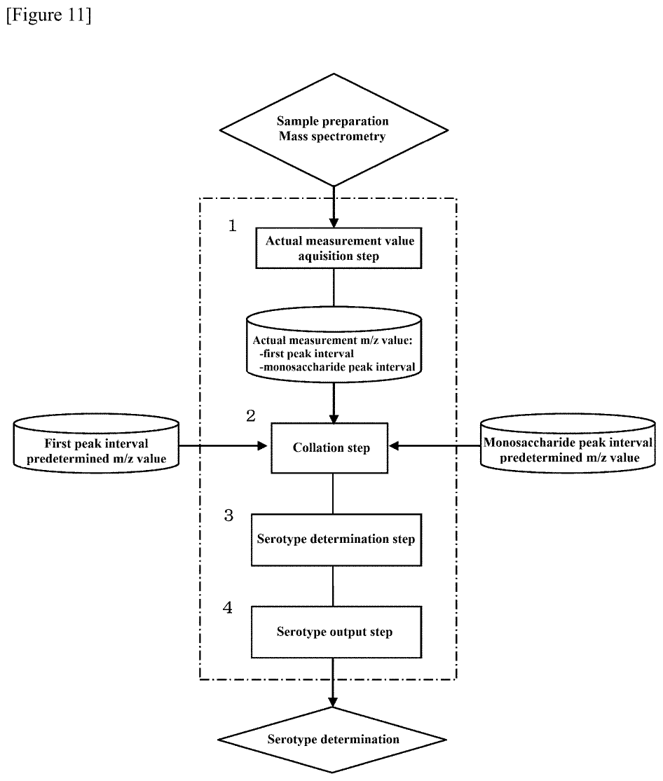

An embodiment of the present invention determination program can be represented by a flowchart as shown in FIG. 11, for example.

By executing the program of step 1, the intervals of successive equally spaced repeating peaks observed in the spectra obtained by the mass spectrometry means are acquired by the data processing means as measured values of m/z, and the data is stored in an appropriate storage medium, if necessary. In step 1, the m/z value of the peak derived from the molecular weight of the monosaccharide composing the unit and the m/z value of the peak interval are also acquired, and the data is stored in the storage medium as necessary.

Those included in the above-mentioned mass spectrometry means are as described above. Examples of the data processing means can include a processor such as a CPU and an MPU. Appropriate storage mediums can include a magnetic recording device (hard disk or the like), an optical disk (CD, DVD, Blu-ray (registered trademark), or the like), a magneto-optical recording medium, and a semi-conductor memory (flash memory, DRAM, SRAM, or the like), for example.

By executing the program of step 2, the measured value and a predetermined value of m/z obtained separately for the repeating peak interval corresponding to a specific serotype are collated by the data processing means, and the data is stored in an appropriate storage medium as mentioned above as necessary. Then, by executing the program of step 3, the serotype related to the predetermined value which matches the measured value among the separately obtained predetermined values is determined to be the serotype of the microorganism by the data processing means, and the data is stored in the storage medium as mentioned above as necessary. Steps 2 and 3 may be performed in parallel.

In step 2 or 3, a program to collate the size of peak intervals derived from monosaccharides can also be incorporated to increase the accuracy of serotype determination.

By executing the program of step 4, the determined serotype of the microorganism is output by an appropriate output means. Such output may be provided to a display, printer, suitable storage medium, or another computer via a telecommunications line (e.g., the Internet).

The present invention determination program may further include the following steps of 5 and/or 6:
5. a step of collating a peak interval actual measurement m/z value between a peak of the peak group consisting of a plurality of monosaccharide molecular weight-derived peaks detected between successive equally spaced repeating peaks and the repeating peak adjacent thereto or the monosaccharide molecular weight-derived peak, with a peak interval predetermined m/z value separately obtained for a detected peak group derived from molecular weights of monosaccharides composing the basic structure corresponding to a specific serotype or with a predetermined m/z value separately obtained by calculation for molecular weights of monosaccharides composing the basic structure, and/or
6. a step of adding the collation result as a condition for the serotype determination.

The hardware resources that specifically execute each of steps 5 and 6 are the same as steps 2 and 3, respectively.

The programs of steps 1 to 6 may be independent of each other or may be any combination of two or more units. For example, the program of step 1 and the program of step 2 may be merged into one program. Alternatively, the programs of steps 1 to 6 may be merged into one program.

The microorganism and the serotype targeted by the present invention determination program are the same as those targeted by the present invention determination method, and are as described above.

### EXAMPLE

Hereinafter, the present invention will be described in more detail with reference to Examples, but the present invention is not limited to the following Examples.

### Example 1

In this example, lipopolysaccharide of E. coli was measured by MALDI-TOF MS (AXIMA Performance manufactured by Shimadzu Corporation), and the size of the repeating peak at equal intervals was collated with a value calculated from the molecular structure of the O-antigen site of the lipopolysaccharide for determining the serotype.

As a sample to be serotyped, lipopolysaccharide derived from E. coli O157 was purchased from Fujifilm Wako Pure Chemical Industries, Ltd. (Code No. 129-05461). Although the serotype was known before the test because the purchased sample clarified the origin, even when the serotype was unknown, the operation was exactly the same. Thus, in this example, the serotypes were deemed to be unknown, and the following measurements, data analysis, collation, and the like were executed.

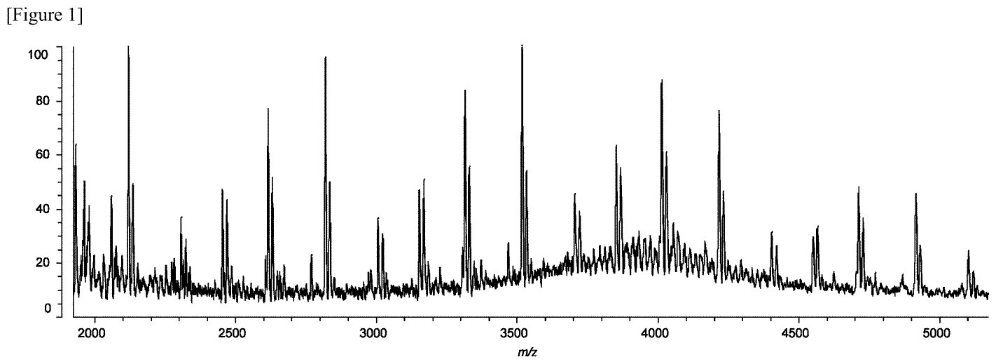

The purchased powdery lipopolysaccharide derived from E. coli O157 was dissolved in purified water and diluted to 0.1 mg/mL (hereinafter referred to as “O157-LPS solution”). 2,5-Dihydroxybenzoic acid was dissolved in a solvent in which purified water and acetonitrile were mixed 1 to 1, and diluted to 2.5 mg/mL (hereinafter referred to as “DHB solution”). O157-LPS solution and the DHB solution were mixed in equal amounts, and 1 μL of the mixed solution was dropped into a target plate for mass spectrometry, dried, and used as a sample for mass spectrometry. This sample was placed in the sample position of the mass spectrometer while being mounted on the target plate (AXIMA Performance manufactured by Shimadzu Corporation), and was measured in the positive ion mode. The measurement resulted in the mass spectrum of FIG. 1.

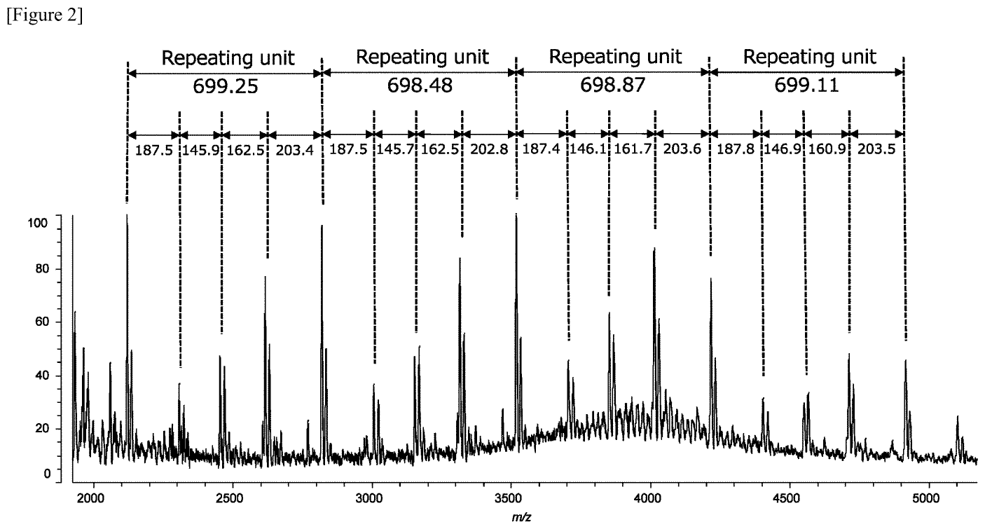

The obtained mass spectrum (FIG. 1) showed periodic repetition. For example, peaks are at m/z 2615.49, m/z 3314.61, m/z 4012.15, m/z 4711.87, and the like, and since the intervals thereof are m/z 699.12, m/z 697.54, and m/z 699.72, these intervals are approximately the same. In addition, peak intervals derived from monosaccharides composing repeating units were also observed among these intervals. For example, the difference between m/z 2615.49 and m/z 2818.22is m/z 202.73, which is considered to be derived from one type of acetylhexosamine. In FIG. 2, a part of the result of calculating the difference between the peaks in this manner is added to the mass spectrum.

Next, among serotypes that sometimes are reported as pathogen of food poisoning caused by enterohemorrhagic Escherichia coli, the sizes of repeating units for O26, O103, O111, and O157 were calculated from the molecular structures of O antigens using a reference document (The structures of Escherichia coli O-polysaccharide antigens., FEMS Microbiol. Rev. 30:382-403.) etc. The results of these calculations are summarized in Table 1 below.

TABLE 1

| | Size | Composition of the repeating unit | | |
|---|---|---|---|---|
| | of the | | Molecular | |
| | repeating | | weight | Num- |
| Serotypes | unit | Monosaccharides | (dehydrated) | ber |
| O26 | 536.5 | rhamnose | 146.1412 | 1 |
| | | N-acetylfucosamine | 187.1931 | 1 |
| | | N-acetylglucosamine | 203.1925 | 1 |
| O103 | 1003.0 | glucose | 162.1406 | 1 |
| | | N-acetylgalactosamine | 203.1925 | 1 |
| | | N-acetylglucosamine | 203.1925 | 2 |
| | | 3-deoxy-3-(R)-3- | 231.2457 | 1 |
| | | hydroxybutyramido-d- | | |
| | | fucose | | |
| O111 | 787.8 | colitose | 130.1418 | 2 |
| | | galactose | 162.1406 | 1 |
| | | glucose | 162.1406 | 1 |
| | | N-acetylglucosamine | 203.1925 | 1 |
| O157 | 698.7 | fucose | 146.1412 | 1 |
| | | glucose | 162.1406 | 1 |
| | | N-acetylperosamine | 187.1931 | 1 |
| | | N-acetylglucosamine | 203.1925 | 1 |

As also shown in FIG. 2, the repeating unit of this sample was roughly of m/z 699 and almost the same as the value of O157 of Table 1. Therefore, the serotype of this sample can be determined to be O157. In FIG. 1 or FIG. 2, a large number of peak intervals reflecting the monosaccharide constituting the repeating unit were observed. For example, the intervals of the peaks at m/z 2119.67, m/z 2307.10, m/z 2615.49, and m/z 2818.92 in FIG. 2 were m/z 187.5, m/z 145.9, m/z 162.5, and m/z 203.4, which were consistent well with the molecular weights (dehydrateds) of the sugars composing the repeating unit in O157 of Table 1. From this, it was also possible to determine that the serotype of the sample in this example was O157.

### Example 2

In this example, E. coli lipopolysaccharides were measured with a matrix-assisted laser desorption ionization-time-of-flight mass spectrometer, and the spectra were compared with the spectra of specimens of known serotypes to determine serotypes.

As a sample to be serotyped, a lipopolysaccharide derived from E. coli O111 was purchased from Sigma, Inc. (code No. L3023). As samples of known serotypes to be compared, lipopolysaccharide derived from E. coli O157 was purchased from Fujifilm Wako Pure Pharmaceutical Co., Ltd. (Code No. 129-05461), lipopolysaccharide derived from E. coli O111 was purchased from Millipore Corporation (Code No. 437627), and lipopolysaccharide derived from E. coli O103 was purchased from Fujifilm Wako Pure Pharmaceutical Co., Ltd. (Code No. 126-05471). Although the serotypes were known before the test because the purchased samples clarified the origins, even when the serotype was unknown, the operation was exactly the same. Thus, in this example, the serotypes were deemed to be unknown, and the following measurements, data analysis, collation, and the like were executed.

The samples used in this example are summarized in Table 2 below.

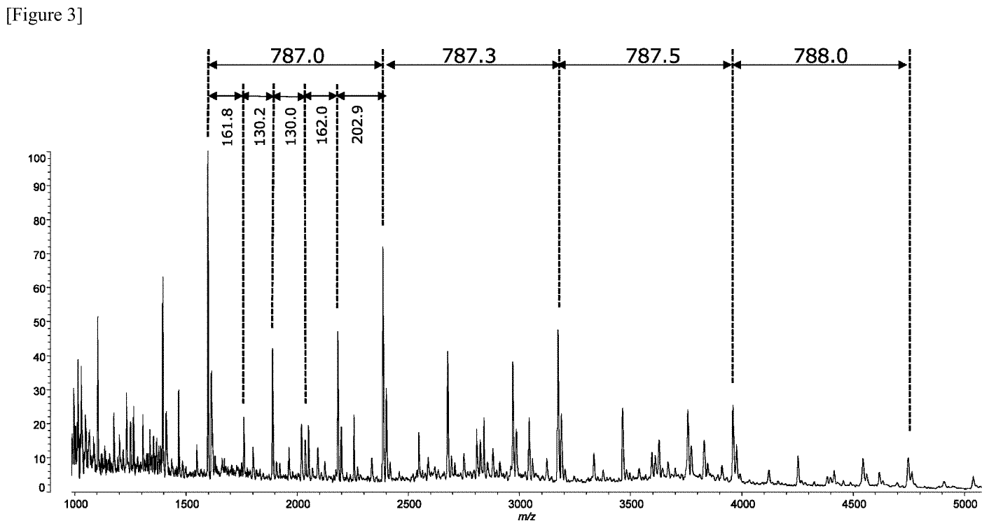

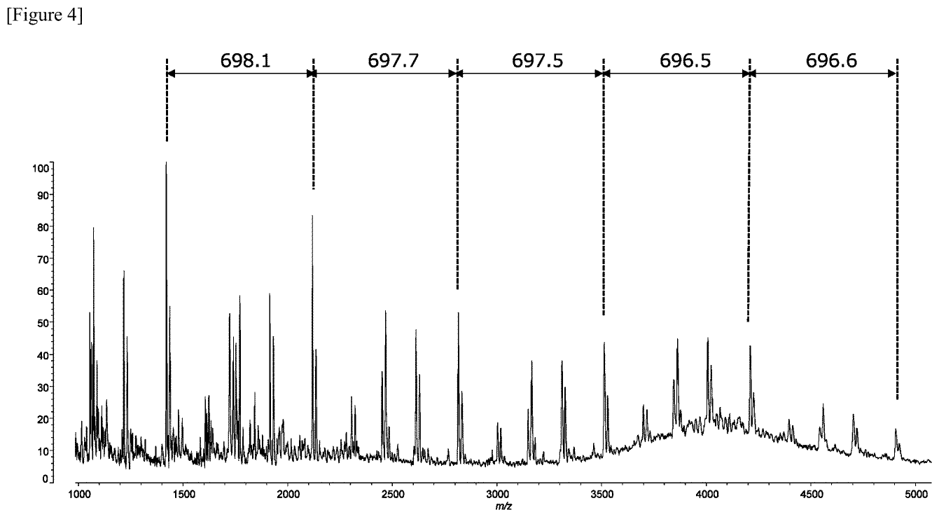

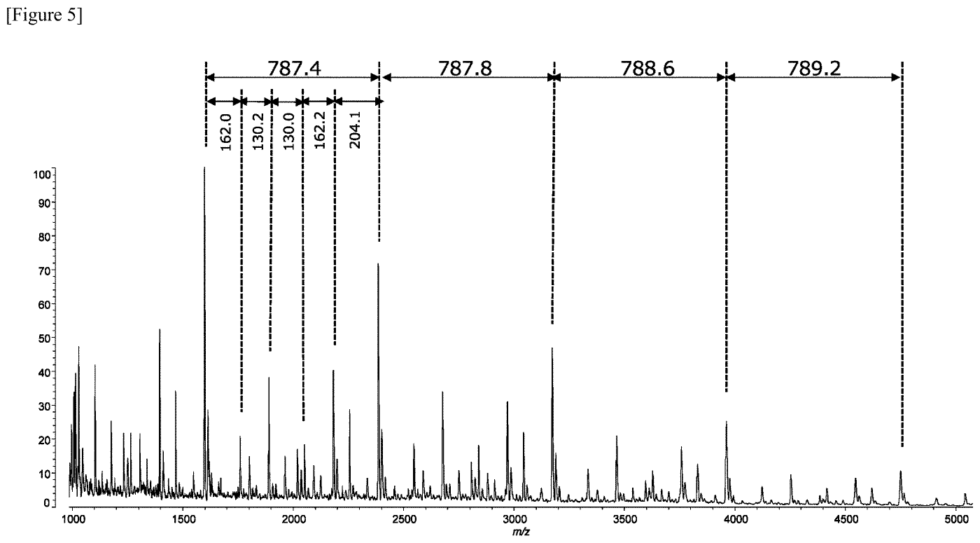

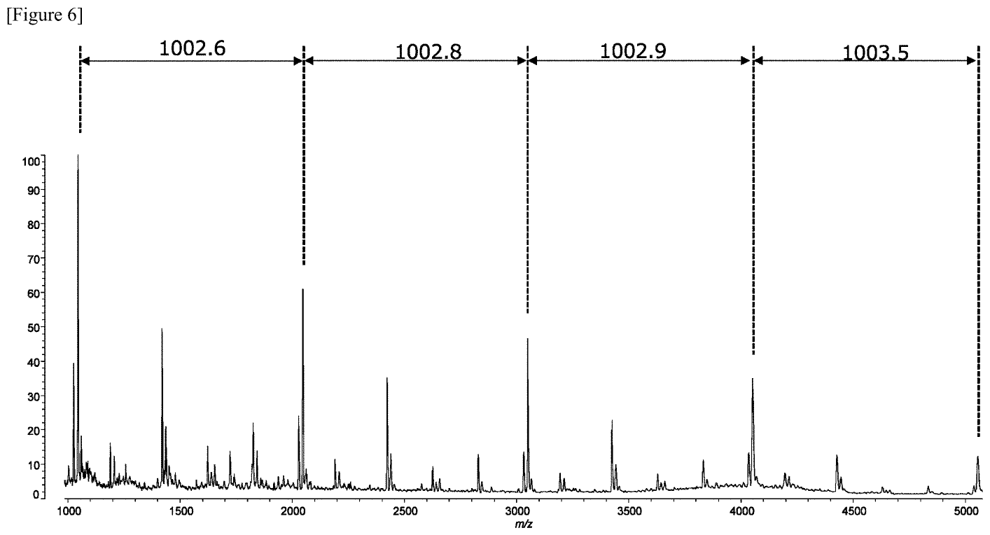

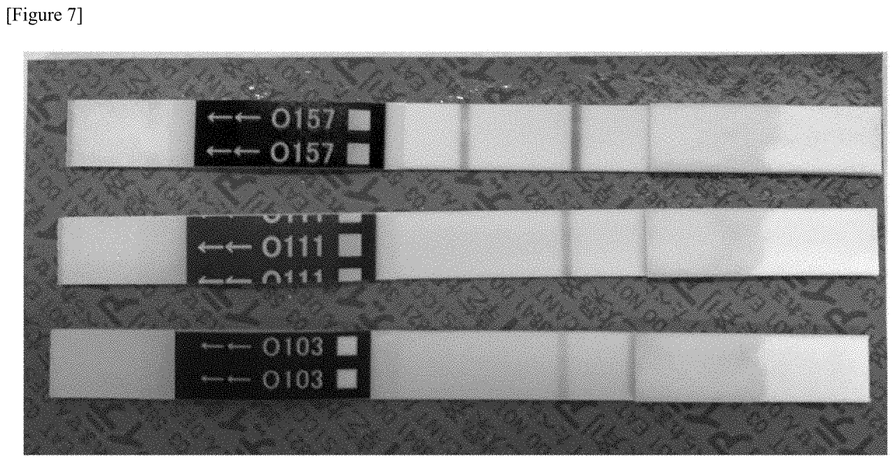

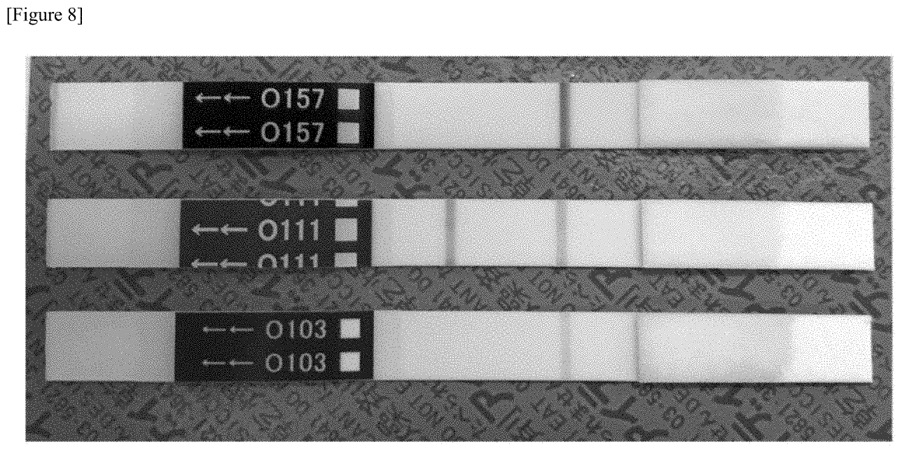

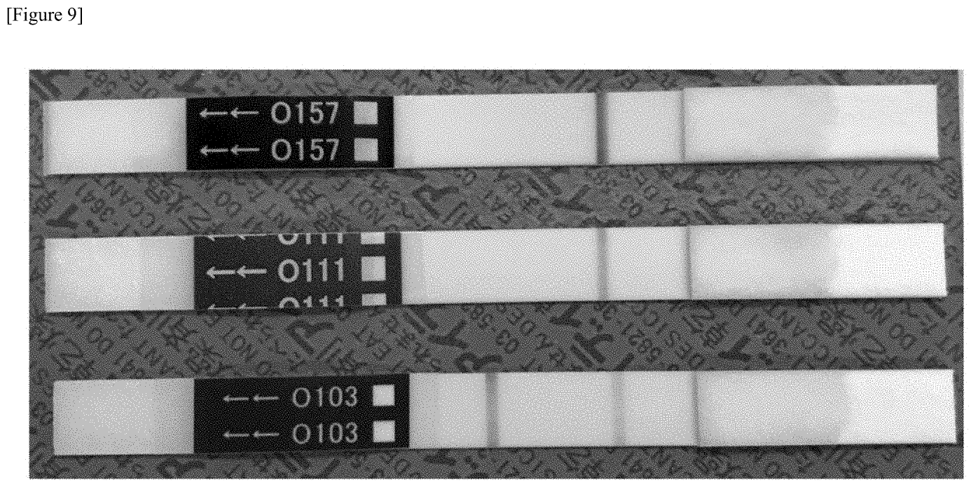

TABLE 2

| Expression | | Results | Results of |
|---|---|---|---|
| in the | | of mass | immunochro- |
| Examples | Maker, code | spectrometry | matography |
| Sample | Sigma Co., L3023 | FIG. 3 | |
| Reference | FUJIFILM Wako Pure | FIG. 4 | FIG. 7 |
| sample A | Chemical., 129-05461 | | |
| Reference | Millipore Co., 437627 | FIG. 5 | FIG. 8 |
| sample B | | | |
| Reference | FUJIFILM Wako Pure | FIG. 6 | FIG. 9 |
| sample C | Chemical., 129-05461 | | |

In this example, the sample, the reference sample A, the reference sample B, and the reference sample C were prepared so as to have a concentration of 0.1 mg/mL by dissolution and dilution with purified water, or the like. 2,5-Dihydroxybenzoic acid was dissolved in a solvent in which purified water and acetonitrile were mixed 1 to 1 and diluted to 2.5 mg/mL. The solutions of the 4 samples said above were mixed in equal amounts with a solution of the 2,5-dihydroxybenzoic acid described above, respectively, and the mixed solution was dropped into a target plate for mass spectrometry by 1 82 L, dried, and used as a sample for mass spectrometry. These samples were placed in the sample position of the mass spectrometer while being mounted on the target plate (AXIMA Performance manufactured by Shimadzu Corporation), and measured in the positive ion mode. By this measurement, the mass spectrum of the sample as shown in FIG. 3, the mass spectrum of the reference sample A as shown in FIG. 4, the mass spectrum of the reference sample B as shown in FIG. 5, and the mass spectrum of the reference sample C as shown in FIG. 6 were obtained, respectively.

From FIG. 3, it can be found that the size of the repeating units of the sample is m/z 788. Similarly, from FIG. 4, it can be found that the repeating unit of the reference sample A is of m/z 698, from FIG. 5, the repeating unit of the reference sample B is of m/z 788, and from FIG. 6, the repeating unit of the reference sample C is of m/z 1,002.

From these results, it was found that the size of the repeating unit of the sample was the same as that of the reference sample B. Moreover, since the peak intervals derived from the constituent sugars in the repeating unit were also the same, it could be found that the subject and the reference sample B have the same structure of the repeating unit portion, that is, the same serotype.

Next, three reference samples were tested with the conventional method, immunochromatography. The result of the reference sample A is shown in FIG. 7, the result of the reference sample B in FIG. 8, and the result of the reference sample C in FIG. 9, respectively. From these results, it was found that the serotype of the reference sample A was O157, the reference sample B was O111, and the reference sample C was O103.

From the above results, as a result of comparing the mass spectrum and the size of the peak interval of the mass spectrum, the sample was of the same serotype as that of the reference sample B. The serotype of reference sample B is O111 determined by immunochromatography. Therefore, the serotype of the sample could be determined to be O111.

### Example 3

By the present invention determination method, those that were determined to have the same O111 serotype by the immunochromatographic method were demonstrated to have different O-antigen sugar chain structures.

The reported O111 sugar chain structures is shown in the upper part of Table 3.

TABLE 3

| | Size | Composition of the repeating unit | | |
|---|---|---|---|---|
| | of the | | Molecular | |
| | repeating | | weight | Num- |
| Serotypes | unit | Monosaccharides | (dehydrated) | ber |
| O111 | 787.8 | colitose | 130.1418 | 2 |
| | | galactose(hexsose) | 162.1406 | 1 |
| | | glucose(hexsose) | 162.1406 | 1 |
| | | N-acetylglucosamin(acetyl- | 203.1925 | 1 |
| | | hexosamine) | | |
| O111* | 828.8 | colitose | 130.1418 | 2 |
| | | acetylhexosamine | 203.1925 | 2 |
| | | hexsose | 162.1406 | 1 |

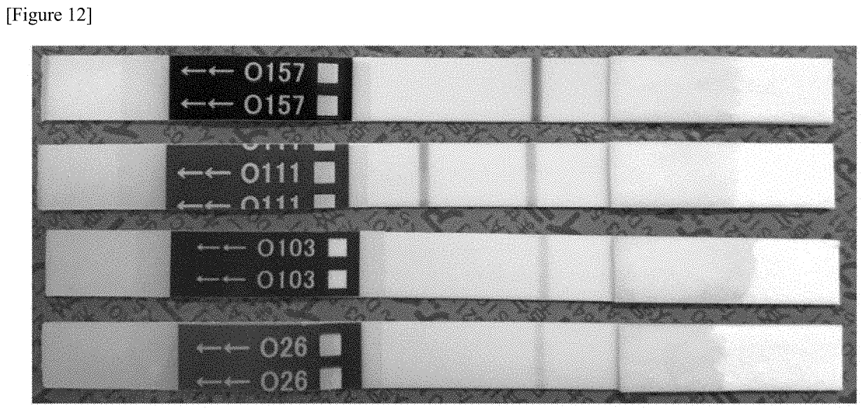

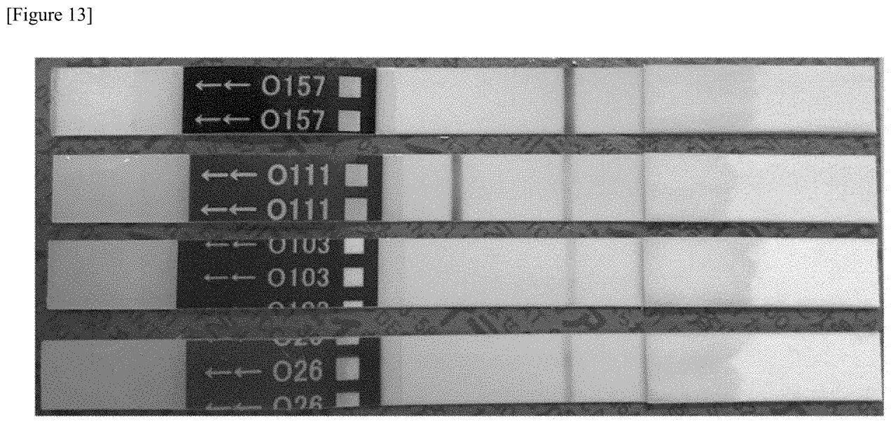

FIG. 12 and FIG. 13 are the results of those that have been previously reported to be O111 by immunochromatographic method, in both of which the test-lines are colored only for O111 strips.

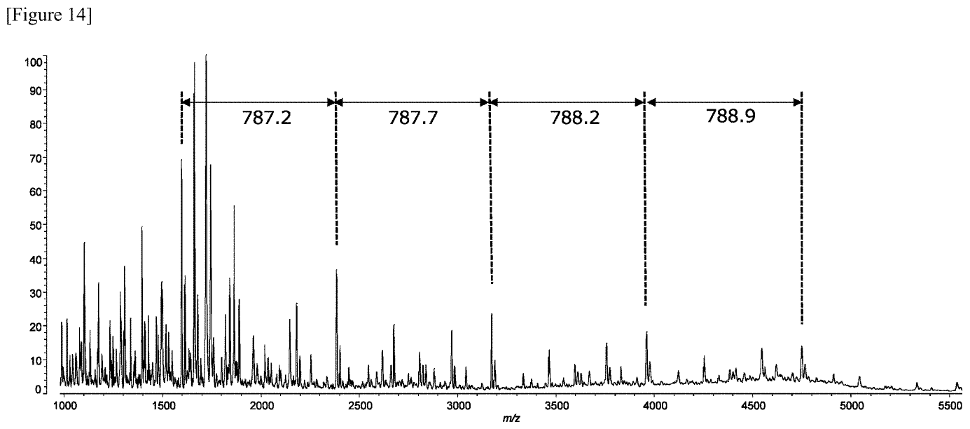

Upon mass spectrometry analysis of E. coli of FIG. 12, the mass spectrum as shown in FIG. 14 was obtained, in which the repeating peaks at intervals of 788 m/z were observed. This was the peak-to-peak interval expected from the structure and was judged to be O111.

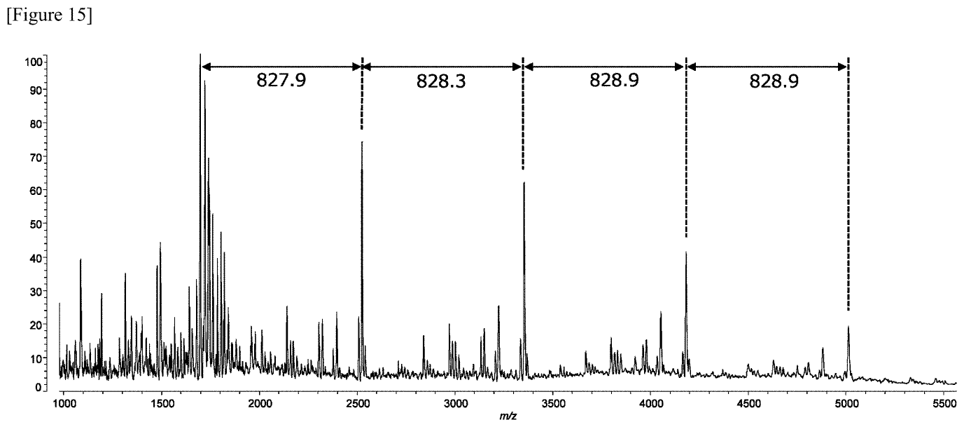

On the other hand, when the E. coli of FIG. 13 was subjected to mass spectrometry, the mass spectrum as shown in FIG. 15 was obtained, and the repeating peaks at intervals of 829 m/z were observed. This was not the peak-to-peak interval expected from the structure, and was judged to be O111* as shown in the lower part of Table 3, which differs from O111 shown in the upper part of Table 3.

As mentioned above, immunochromatography showed that both O111 and O111* exhibited the same reaction (outcome), but the mass spectrum by mass spectrometry demonstrated that O111 and O111* are different in the structure of the O antigen sugar chain. According to the present invention determination method, it was estimated that in O111, two hexoses and 1 acetylhexosamine are present, whereas in O111*, one hexose and two acetylhexosamine are present.

The similar outcome of O111 and O111* in immunochromatography may be attributed to the used antibody recognizing the common structural part in both materials.

1. A method of determining a serotype of a microorganism wherein a sugar chain moiety of a lipopolysaccharide forming an antigen is repeated multiple times with a basic structure composed of a combination of a plurality of monosaccharides as one unit, comprising the following steps 1 to 3:
1) a step of selecting a first peak group consisting of a plurality of peaks which are successively and repeatedly detected at equal intervals in a spectrum obtained by performing a mass spectrometry on a sample containing an antigenic site obtained from a microbial specimen of a determination subject, and then obtaining a difference in m/z value between adjacent peaks of the first peak group as a peak interval actual measurement m/z value of the first peak group,
2) a step of collating the peak interval actual measurement m/z value of the first peak group with a separately obtained m/z value for the basic structure corresponding to a specific serotype, and
3) a step of determining the specific serotype corresponding to the separately obtained m/z value for the basic structure consistent with the peak interval actual measurement m/z value of the first peak group, as the serotype of the microorganism, from the result of the collation step.

2. The serotype determination method according to claim 1, wherein the separately obtained m/z value for the basic structure corresponding to a specific serotype is a value calculated from a plurality of monosaccharides composing the basic structure.

3. The serotype determination method according to claim 1, wherein the separately obtained m/z value for the basic structures corresponding to a specific serotype is an m/z value between adjacent peaks of a peak group consisting of a plurality of peaks detected successively and repeatedly at equal intervals in a mass spectrum obtained by mass spectrometry of a microorganism with a known serotype.

4. The serotype determination method according to claim 1, further comprising a step of collating a peak interval actual measurement m/z value between a peak of a second peak group consisting of a plurality of monosaccharide molecular weight-derived peaks detected between each peak composing the first peak group, and a peak of the first peak group adjacent thereto or a monosaccharide molecular weight-derived peak of the second peak group, with a separately obtained peak interval predetermined m/z value for a detected peak group derived from molecular weights of monosaccharides composing the basic structure corresponding to a specific serotype, or with a peak interval predetermined m/z value separately obtained by calculation for the peak group derived from molecular weights of monosaccharides composing the basic structure, and adding the collating result as a condition for determining a serotype.

5. The serotype determination method according to claim 1, wherein the microorganism is a Gram-negative bacterium or E. coli.

6. The serotype determination method according to claim 1, wherein the serotype is a serotype of an O-antigen.

7. An apparatus for determining a serotype of a microorganism wherein a sugar chain moiety of a lipopolysaccharide forming an antigen is repeated multiple times with a basic structure composed of a combination of a plurality of monosaccharides as one unit, comprising the following units 1 to 4:
1) a data acquisition unit, which acquires a mass spectrum data obtained by performing a mass spectrometry on a sample containing an antigenic site prepared from a microbial specimen of a determination subject,
2) a first peak interval acquisition unit, which selects a first peak group consisting of a plurality of peaks which are successively and repeatedly detected at equal intervals in the mass spectrum obtained in the data acquisition unit, and then obtains a difference in m/z value between adjacent peaks of the first peak group as a peak interval actual measurement m/z value of the first peak group,
3) a data collation unit, which collates the actual measurement m/z value with a separately obtained m/z value for the basic structure corresponding to a specific serotype, and
4) a serotype determination unit, which determines a specific serotype corresponding to the separately obtained m/z value consistent with the actual measurement m/z value, as the serotype of the microorganism.

8. The apparatus for determining a serotype of a microorganism according to claim 7, further comprising a data collation unit, which collates a peak interval actual measurement m/z value between a peak of the peak group consisting of a plurality of monosaccharide molecular weight-derived peaks detected between each peak composing the first peak group and a peak of the first peak group adjacent thereto or a monosaccharide molecular weight-derived peak, with a peak interval predetermined m/z value separately obtained for a detected peak group derived from molecular weights of monosaccharides composing the basic structure corresponding to a specific serotype or with a peak interval predetermined m/z value separately obtained by calculation for the peak group derived from molecular weights of monosaccharides composing the basic structure, and/or
a serotype determination unit, which can add the collation result as a condition for the serotype determination.

9. The apparatus for determining a serotype of a microorganism according to claim 7, wherein the microorganism is a Gram-negative bacterium or E. coli.

10. The apparatus for determining a serotype of a microorganism according to claim 7, wherein the serotype is a serotype of an O antigen.

11. A non-transitory computer-readable medium including a program for determining a serotype of a microorganism wherein a sugar chain moiety of a lipopolysaccharide forming an antigen is repeated multiple times with a basic structure composed of a combination of a plurality of monosaccharides as one unit, executing the steps comprising the following 1 to 4:
1) a step of performing a mass spectrometry on a sample containing an antigenic site prepared from a microbial specimen of a determination subject, and then acquiring an interval between each adjacent peak of a plurality of peaks detected successively and repeatedly at equal intervals in a spectrum obtained therein as an actual measurement m/z value,
2) a step of collating the actual measurement m/z value with a separately obtained m/z value for the basic structure corresponding to a specific serotype,
3) a step of determining the specific serotype corresponding to the separately obtained m/z value consistent with the actual measurement m/z value, as the serotype of the microorganism, and
4) a step of outputting the determined serotype of the microorganism.

12. The non-transitory computer readable medium including the program for determining a serotype of a microorganism according to claim 11, further comprising a step of collating a peak interval actual measurement m/z value between a peak of the peak group consisting of a plurality of monosaccharide molecular weight-derived peaks detected between successive equally spaced repeating peaks and the repeating peak adjacent thereto or the monosaccharide molecular weight-derived peak, with a peak interval predetermined m/z value separately obtained for a detected peak group derived from molecular weights of monosaccharides composing the basic structure corresponding to a specific serotype or with a predetermined m/z value separately obtained by calculation for the peak group derived from molecular weights of monosaccharides composing the basic structure, and/or a step of adding the collation result as a condition for the serotype determination.

13. The non-transitory computer readable medium including the program for determining a serotype of a microorganism according to claim 11, wherein the microorganism is a Gram-negative bacterium or E. coli.

14. The non-transitory computer readable medium including the program for determining a serotype of a microorganism according to claim 11, wherein the serotype is a serotype of an O antigen.
